(19) United States
(12) Patent Application Publication
(10) Pub. No.: US 2018/0318140 A1
(43) Pub. Date: Nov. 8, 2018

(54) Buffer for Offloading Cast

(71) Applicant: Stephen Valiakalayil Pappachen, Lakeland, FL (US)

(72) Inventor: Stephen Valiakalayil Pappachen, Lakeland, FL (US)

(21) Appl. No.: 15/587,405

(22) Filed: May 5, 2017

Related U.S. Application Data
(66) Substitute for application No. 29/600,143, now abandoned.

(51) Int. Cl. A61F 13/04; A61D 9/00

(52) U.S. Cl. CPC A61F 13/041; A61D 9/00

(57) ABSTRACT

A bubble-wrap type layer of protection either in addition to or in place of any existing buffer design will greatly enhance the offloading as well as protective effects of any offloading cast.

### OVERVIEW

1. A bubble-wrap type layer, which can have the consistency of plastic, rubber or any other non-abrasive substance/material, will be manufactured according to existing specifications with care taken not to infringe on any existing patents and/or purchased.
2. Subsequently, the bubble wrap material will be used either in replacement of existing buffer material or as an adjunct to it. Either way, no claim will be made infringing on the design or utility of any offloading cast itself (e.g. fiberglass and/or exterior hard shell/cover) currently in existence. Rather, the buffer material used to wrap the relevant anatomy/extremity is the subject of this specification and any subsequent claim.
3. The bubble wrap material can either be manufactured/used as a sheet and folded over/appropriately to fit the contour of the anatomy and/or manufactured specifically for use on/around a particular area of the anatomy. The anatomy doesn't necessarily have to be human (e.g. relevant veterinary applications would also be covered under this specification and any subsequent claim.
4. Any variation of number 3 above—as relevant to the application/use of the bubble wrap material—would be covered under this specification.

### FIGURES

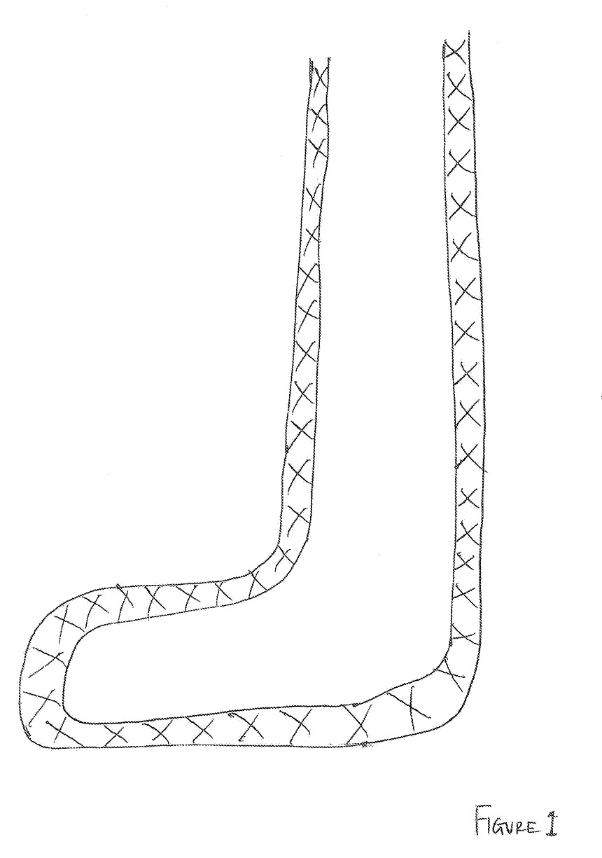

FIG. 1:
1. This graphic represents an offloading cast, which is not the subject of my claim or my invention.
2. The offloading cast is used to relieve the pressure from a wound site in the affected lower extremity. The offloading/pressure relieving modality helps redistribute/relieve the direct impact of pressure on the wound site and, thus, promotes healing of the wound.
3. However, in relieving/re-distributing pressure from a wound site; excess or unequal pressure can be transferred to other wound sites, or area of the body that are either normal (e.g. no wound) or at risk for wounds (e.g. pre-wound areas).
4. The buffer material for the offloading device, therefore, becomes a key element of the discussion on how to prevent and protect wounded or non-wounded areas of the body that subject to the offloading modality.

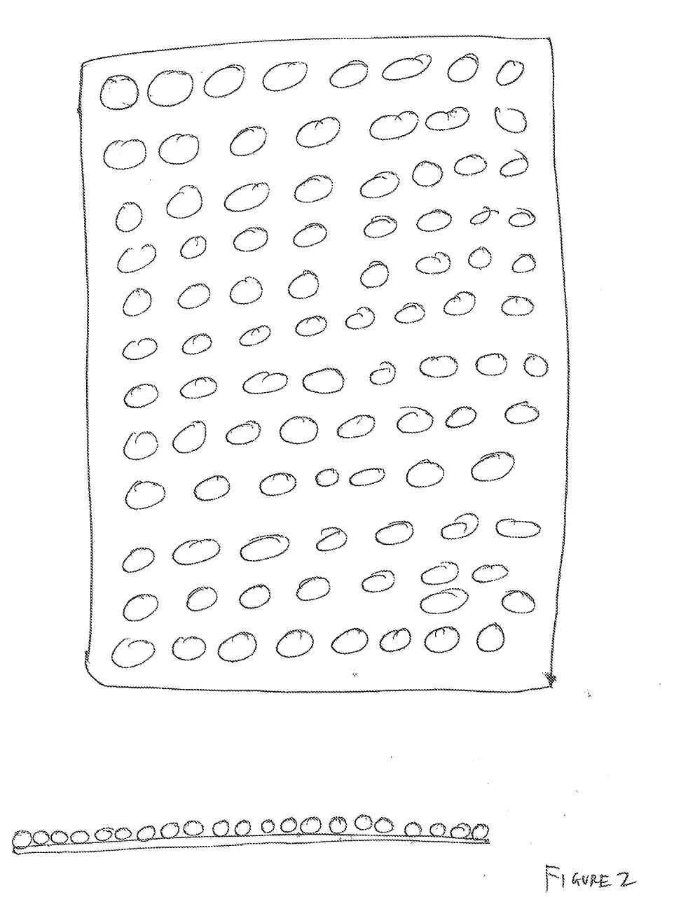

FIG. 2:
1. This graphic represents material known as bubble-wrap, ii would propose that bubble wrap is a great offloading buffer. This notion/idea represents part of my claim.
2. The idea for using different types of bubble wrap made for the specific purpose of being used as a buffer in offloading devices such as the total contact cast represents my claim as well.
3. I propose that bubble wraps (different kinds and different types) used either by itself or in conjunction with other types of offloading buffers can provide excellent offloading and provides superior protection for wounded and non-wounded areas.

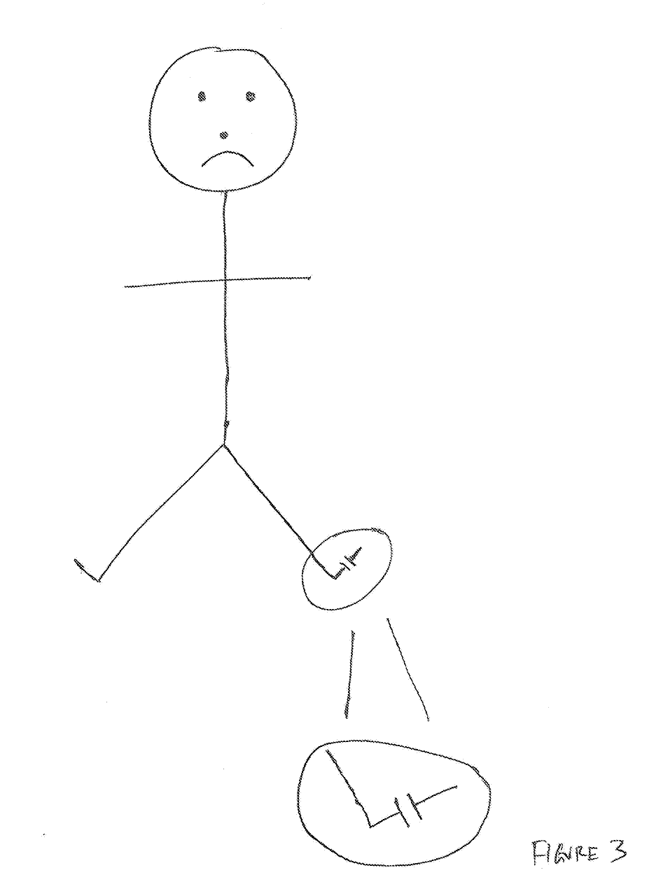

FIG. 3:
1. This graphic represents a patient with a wound/ulcer.
2. As you can clearly see, this individual is not happy, They're not happy because they have a wound and all the things that go along with having a wound in their lower extremity (e.g, pain, possibly infection, inconvenience, etc. . . . ).

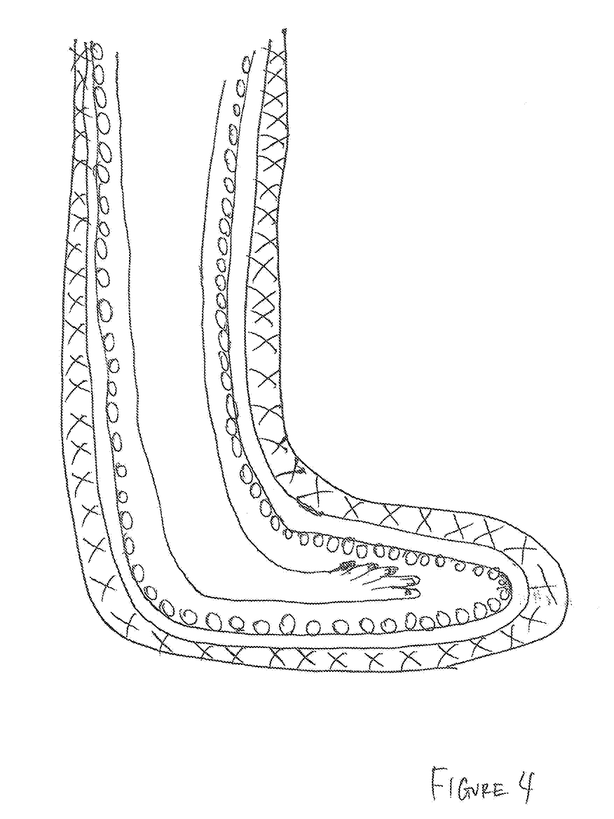

FIG. 4:
1. This graphic represents placement of the previous patient's lower extremity (including wounded area) in an offloading cast with my bubble wrap as the buffer material.
2. As you can hopefully see, the lower extremity (including wounded and non-wounded areas) are being offloaded (undergoing pressure relief) and well protected.

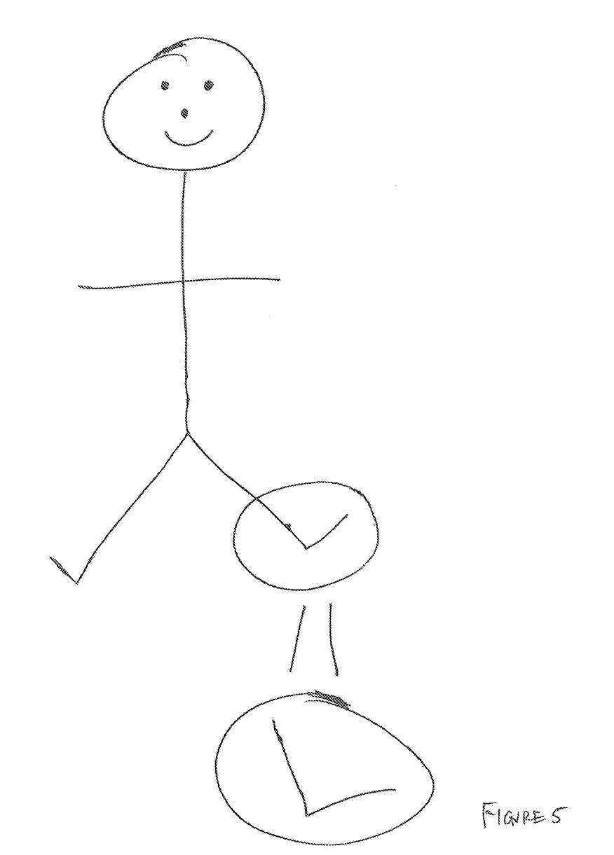

FIG. 5:
1. This graphic represents the same patient after they have undergone the proper offloading with resultant wound healing.
2. Clearly, this patient is much happier. What makes this scenario possible is the proper offloading buffer (e.g. my bubble wrap idea) that helps offload the wound and prevent new wounds from developing.

1. A bubble-wrap type layer, which can have the consistency of plastic, rubber or any other non-abrasive substance/material, will be manufactured according to existing, specifications with care taken not to infringe on any existing patents and/or purchased. As far as know, this kind of use/utility has never been patented.

2. Subsequently, the bubble wrap material will be used either in replacement of existing buffer material or as an adjunct to it. Either way, no claim will be made infringing on the design or utility of any offloading cast itself (e.g. fiberglass and/or exterior hard shell/cover) currently in existence. Rather, the buffer material used to wrap the relevant anatomy/extremity is the subject of this specification and any subsequent claim. As far as I know, this kind of use/utility has never been patented.

3. The bubble wrap material can either be manufactured/used as a sheet and folded over/appropriately to fit the contour of the anatomy and/or manufactured specifically for use on/around a particular area of the anatomy. The anatomy doesn't necessarily have to be human (e.g. relevant veterinary applications would also be covered under this specification and any subsequent claim. As far as I know, this kind of use/utility has never been patented.

4. Any variation of number 3 above—as relevant to the application/use of the bubble wrap material—would be covered under this specification. As far as I know, this kind of use/utility has never been patented.
